(19) United States
(12) Patent Application Publication
(10) Pub. No.: US 2025/0152791 A1
(43) Pub. Date: May 15, 2025

(54) Connector and breast pump with the same

(71) Applicant: Dongguan Rongmao Medical Technology Co., Ltd., Dongguan (CN)

(72) Inventor: Yongqiang ZHANG, Dongguan (CN)

(21) Appl. No.: 18/675,629

(22) Filed: May 28, 2024

(30) Foreign Application Priority Data
Nov. 13, 2023 (CN) 202311512360.5

(51) Int. Cl. A61M 1/06; A61M 39/10

(52) U.S. Cl. CPC A61M 1/069; A61M 39/10

(57) ABSTRACT

A connector includes a connecting barrel with a front opening and a rear opening opposite to each other, a periphery of the rear opening extends outward to form a rear chamber, the connecting barrel is in communication with the rear chamber, a bottom surface of the connecting barrel is provided with a first through-hole, and the rear chamber is provided with a second through-hole.

### TECHNICAL FIELD

The present disclosure relates to the technical field of breast pumps, more particularly, a connector used for a breast pump and a breast pump with the connector.

### BACKGROUND

A breast pump mainly comprises a motor, a three-way assembly, and a milk storage chamber. The working principle is that the motor drives the three-way assembly to simulate a sucking action so that the mother milk enters the milk storage chamber to complete the collection of the mother milk. The existing electric breast pump is divided into two types: a split-type and an integrated type. In the split type breast pump, a motor and a three-way assembly are separate, a motor is external and connected to the three-way assembly via a long hose, and the three-way assembly is communicated with the milk storage chamber. The external host has high power, which can achieve the high-efficiency collection of mother milk. However, the split-type breast pump has a large volume for storage and is inconvenient to carry and use. However, in the integrated breast pump, the main machine, the three-way assembly, and the milk storage chamber are integrated arrangement, the integrated breast pump is small and convenient to carry and can be used by mom to collect mother's milk at any time, and can be worn and used without being held by hands.

Chinese patent CN116650742A discloses a breast pump, comprising a housing, a breast-adhering cover, a negative-pressure assembly, and a deformation suction portion; the housing is provided with a milk storage cavity and a negative pressure cavity, and the breast-adhering cover comprises a flange for fitting with a breast and a passage suitable for placing a breast, the passage is in communication with the milk storage cavity, and the negative pressure assembly is used for providing a negative pressure to the negative pressure cavity, the deformation suction portion is hermetically connected to an opening of the negative pressure cavity, and transmits the negative pressure provided by the negative pressure assembly to the breast-adhering cover, so that one side of the deformation suction portion is in communication with the negative pressure cavity.

Chinese utility model CN 218833270 U discloses a breast pump, comprising a milk storage bottle, a suction silicone bowl, a silicone flange, and a host machine; the suction silicone bowl is mounted inside the milk storage bottle, a side wall of the suction silicone bowl is provided with a one-way valve, the suction silicone bowl is provided with a milk collecting cavity and a negative pressure cavity, which are separated by a deformation wall of the suction silicone bowl; an outer periphery of the silica gel flange is closely connected to the milk storage bottle; a central portion of the silica gel flange is closely connected to the suction silica gel bowl; the central portion is provided with a collecting hole in communication with the milk collecting cavity; an inner wall of the milk storage bottle and an inner wall of the silica gel flange enclose a milk storage cavity. The main machine is mounted on the periphery of the milk storage bottle, a suction end of the main machine is in communication with the negative pressure cavity, and the main machine is used for making the negative pressure cavity form a negative pressure, so that the deformable wall can move back and forth between the milk collecting cavity and the negative pressure cavity.

However, there are many parts of the breast pump, a negative pressure cavity, a milk storage bottle, a silica gel flange, a main machine, and a housing, and other parts therebetween all need to be provided with structures capable of matching and connecting, so that the whole breast pump can be installed, and the structure is complex and the installation is complex. In particular, in order to control the negative pressure cavity by the negative pressure pump to generate positive pressure and negative pressure, so as to promote the breast to secrete milk, the requirement for the connection and sealing between some parts is high, and a scaling structure needs to be additionally provided.

The milk channel of the existing wearable breast pump is long and occupies too much space of the breast pump, resulting in an increased thickness of the breast pump, which is uncomfortable to wear. In addition, the wearable breast pump is mainly used in a public place or a workplace in order to facilitate a career's mother. If the breast pump is too thick, the chest of the career mother will protrude too far if being worn, which not only affects the beauty but also cannot protect privacy. Moreover, various components of the breast pump are distributed unevenly, and the breast can be easily displaced and dragged during wearing, thereby affecting the health of the breast.

In addition, the existing wearable breast pump, the milk inlet of the milk storage chamber is provided at the bottom thereof. During use, milk enters the milk storage chamber from the bottom thereof, and the existing milk in the milk storage chamber needs to be pushed upwards. When the gravity of the existing milk in the milk storage chamber is greater than the pushing power, the milk cannot enter the milk storage chamber, thereby indirectly reducing the secretion of breast milk.

### SUMMARY

The present disclosure aims to solve the technical problem that “the breast pump is too thick” and “the weight distribution of various components of the breast pump is uneven” in the existing technology. Therefore, the present disclosure provides a connector and a breast pump with the same, which can integrate a trumpet-shaped cavity, a negative pressure chamber, a host module, and a storage chamber in front, behind, above, and below the connector respectively, optimize the structure of the breast pump, reduce the thickness of the breast pump, and make the distribution of various components uniform.

A connector of a breast pump includes a connecting barrel with a front opening and a rear opening opposite to each other, a periphery of the rear opening extends outward to form a rear chamber, and the connecting barrel is in communication with the rear chamber. The bottom surface of the connecting barrel is provided with a first through-hole, and the rear chamber is provided with a second through-hole.

The rear chamber includes a corrugated plate connecting the rear opening, a side wall perpendicularly extends from the periphery of the corrugated plate such that the rear chamber is in a groove shape; the second through-hole is formed in a top portion of the side wall.

A breast pump having the above connector includes a front shell and a rear shell which are connected in a matching manner, a front side face of the front shell is provided with a trumpet-shaped cavity, and the front opening faces the trumpet-shaped cavity.

The connector is provided in the middle of the rear shell and integrally formed with the rear shell.

The rear shell has a rear-shell left portion, a rear-shell right portion, and a rear-shell middle portion connecting the rear-shell left portion and the rear-shell middle portion; the connector is provided in the rear-shell middle portion and integrally formed with the rear shell; the side wall of the rear chamber connects the rear-shell middle portion; the rear-shell left portion and the rear-shell right portion are located at two sides of the connecting barrel.

The rear-shell middle portion is provided with an empty place at a position directly facing the rear chamber.

A first partition and a second partition extend symmetrically from the left and right side walls of the connecting barrel, the first partition is connected to the rear-shell left portion, and the second partition is connected to the rear-shell right portion so that the rear shell is divided into an upper part of the rear shell and a lower part of the rear shell.

The trumpet-shaped cavity passes through the front shell with the small opening end thereof connecting the front opening of the connecting barrel. The front shell has a front-shell left portion, a front-shell right portion, and a front-shell middle portion. The trumpet-shaped cavity is located in the front-shell middle portion. The front shell extends from the left and right sides of the small opening end of the trumpet-shaped cavity to form a front-left edge connecting the front-shell left portion and a front-right edge connecting the front-shell right portion respectively. The front-left edge connects the first partition and the front-right edge connects the first second partition such that the inner cavity of the breast pump is divided into an upper mounting chamber and a lower storage chamber.

The sealing ring is provided at the joint of the front shell and the rear shell, connecting the front-left edge and the first partition, the front-right edge and the second partition, and the small opening end and the connecting barrel in a sealed manner.

The front shell has a front-shell left portion, the front-shell right portion, and a front-shell middle portion connecting the front-shell left portion and the front-shell right portion. The trumpet-shaped cavity is provided in the front-shell middle portion, and the small opening end of the trumpet-shaped cavity and the front opening of the connecting barrel is integrally formed.

The breast pump further includes a rear shell matching the front shell, the rear shell is provided with a fitting groove at a position directly facing the rear chamber, and the rear chamber is partially accommodated in the fitting groove.

The front shell extends from the left and right sides of the small opening end of the trumpet-shaped cavity to form a front-left edge connecting the front-shell left portion and a front-right edge connecting the front-shell right portion respectively. A first partition and a second partition extend symmetrically from left and right side walls of the fitting groove, the first partition is connected to the rear-shell left portion, and the second partition is connected to the rear-shell right portion, the front-left edge connects the first partition, the front-right edge connects the second partition so that the inner cavity of the breast pump is divided into the upper mounting chamber and the lower storage chamber.

The breast pump has a host module, which is detachably connected to the front shell and the rear shell, and the host module comprises a central driving assembly and a symmetrical power supply assembly, the power supply assembly is electrically connected to the driving assembly, and a suction port of the driving assembly is in communication with a negative pressure chamber arranged in the rear chamber.

The top portion of the front shell or the rear shell is provided with a milk channel, and when the host module, the front shell, and the rear shell are assembled, the bottom portion of the host module shields a port of the milk channel.

The breast pump in the disclosure is provided with an integrated connector, which integrates the host module, the storage chamber, the trumpet-shaped cavity, and the negative pressure cavity in the upper direction, the lower direction, the front direction, and the rear direction of the connector, respectively. A connection and a mounting structure do not need to be additionally provided between each part, thereby greatly simplifying the structure of the breast pump, and facilitating disassembly and mounting.

In the breast pump in the disclosure, the host module, the storage chamber, the trumpet-shaped cavity, and the negative pressure cavity are respectively integrated in the upper direction, the lower direction, the front direction, and the rear direction of the connector, so that the thickness of the breast pump in the front and rear directions is relatively reduced, such that the mother's chest does not protrude excessively after wearing, the breast pump is streamlined as a whole and has an aesthetic appearance.

In the breast pump in the disclosure, the negative pressure chamber and the below silica gel deformation part are disposed right behind the trumpet-shaped cavity, so that the power loss caused by the deformation of the negative pressure chamber is reduced to the greatest extent, the positive and negative pressure change effect in the trumpet-shaped cavity is good, and the lactation efficiency is high.

In the breast pump in the disclosure, the second fitting edge connects the second partition such that the space between the front shell and the rear shell is separated into the upper mounting chamber and the lower storage chamber, and the host module is mounted in the upper mounting chamber. On the one hand, this structure can prevent the milk from entering the mounting chamber to contaminate or damage to the host module, and on the other hand, the host module is arranged on the top of the breast pump, which is very convenient to mount and detach. In addition, the joint of the front shell and the rear shell is provided with the sealing ring. The sealing ring, the first fitting edge, the first partition, the second fitting edge, and the second partition are fitted and separate a milk pathway and a gas pathway of the breast pump. The milk pathway and the gas pathway do not affect each other, and the milk flows smoothly without remaining.

According to some embodiments in the disclosure, the breast pump includes the shell and the host module. The shell has a lower storage chamber, the negative pressure chamber, and the trumpet-shaped cavity, the trumpet-shaped cavity is in unidirectional communication with the lower storage chamber, and the negative pressure chamber is located at one side of the trumpet-shaped cavity.

The host module is detachable with the shell, which includes a driving assembly and a power supply assembly symmetrically arranged on two sides of the driving assembly.

The power supply assembly is electrically connected to the driving assembly, and an air suction port of the driving assembly is in communication with the negative pressure chamber.

The storage chamber is provided with a milk channel extending to the top of the shell, when the host module and the shell are assembled, the bottom of the host module shields the port of the mill channel.

According to some embodiments in the disclosure, a connecting barrel, a trumpet-shaped cavity, and a pad are sequentially arranged in the front shell; the connecting barrel is arranged at one side of the negative pressure chamber; one side of the trumpet-shaped cavity is in communication with the connecting barrel; and the other side thereof is provided with a pad.

According to some embodiments in the disclosure, the contour of the trumpet-shaped cavity is arranged in a breast-like shape, and the pad is arranged on the wall of the trumpet-shaped cavity and is used for attaching to the breast.

According to some embodiments in the disclosure, a silica gel deformation part is disposed between the connecting barrel and the negative pressure chamber, and the surface contour where the shell attaches to the silica gel deformation part and that of the silica gel deformation part match each other.

According to some embodiments in the disclosure, the bottom of the connecting barrel is provided with an unidirectional valve, the connecting barrel is in an unidirectional communication with the storage room.

According to some embodiments in the disclosure, the shell includes the front shell and the rear shell, the trumpet-shaped cavity is located in the front shell, the connecting barrel is located at the rear surface of the front shell, and the storage chamber is formed by a combination of the front shell and the rear shell.

According to some embodiments in the disclosure, the joint of the front shell and the rear shell is arranged with the sealing ring, the seal ring connects the trumpet-shaped cavity, the connecting barrel, and the storage chamber in a sealed manner.

According to some embodiments in the disclosure, the host module is provided with a negative pressure hole. The air suction port of the driving assembly and the negative pressure hole are connected by the silica gel tube and the soft glue pad. When the host module is mounted in the shell, the negative pressure hole and the second through-hole of the negative pressure chamber is connected tightly.

According to some embodiments in the disclosure, the host module includes a control assembly, arranged above the driving assembly and coupled with the driving assembly.

According to some embodiments in the disclosure, the host module and the shell are fastened through an elastic structure.

According to some embodiments in the disclosure, the breast pump has at least the following beneficial effects:
The milk channel of the storage chamber extends directly to the top of the shell, which shortens the milk channel, reduces the space occupied by the milk channel of the storage chamber, and uses the host module to shield the port of the milk channel, effectively reducing the thickness of the breast pump, making it more fitting and comfortable to wear. The driving assembly and the power supply assembly, which have a relatively large weight, are respectively arranged in a central and symmetrical structure, making the weight distribution of the breast pump more uniform. During the wearing process, it is not easy to shift or drag the breasts, protecting breast health.

Additional aspects and advantages of the present disclosure will be given in part in the following descriptions, become apparent in part from the following descriptions, or be learned from the practice of the present disclosure.

### BRIEF DESCRIPTION OF THE DRAWINGS

The foregoing and/or additional aspects and advantages of the present disclosure will become apparent and more readily appreciated from the following descriptions of embodiments taken in conjunction with the accompanying drawings.

FIG. 1 is a structural schematic diagram of a connector of the breast pump according to some embodiment in the present disclosure.

FIG. 2 is a schematic view of the rear shell of the breast pump according to one embodiment in the present disclosure.

FIG. 3 is a schematic view of the rear shell of the breast pump according to another embodiment in the present disclosure.

FIG. 4 is a schematic diagram of the structure of the breast pump according to some embodiments in the present disclosure.

FIG. 5 is a schematic exploded structural view of the breast pump according to some embodiments in the present disclosure.

FIG. 6 is a sectional view of the breast pump according to some embodiments in the present disclosure.

FIG. 7 is another sectional view of the breast pump according to some embodiments in the present disclosure.

FIG. 8 is an exploded structural schematic view from another angle of the breast pump according to some embodiments in the present disclosure.

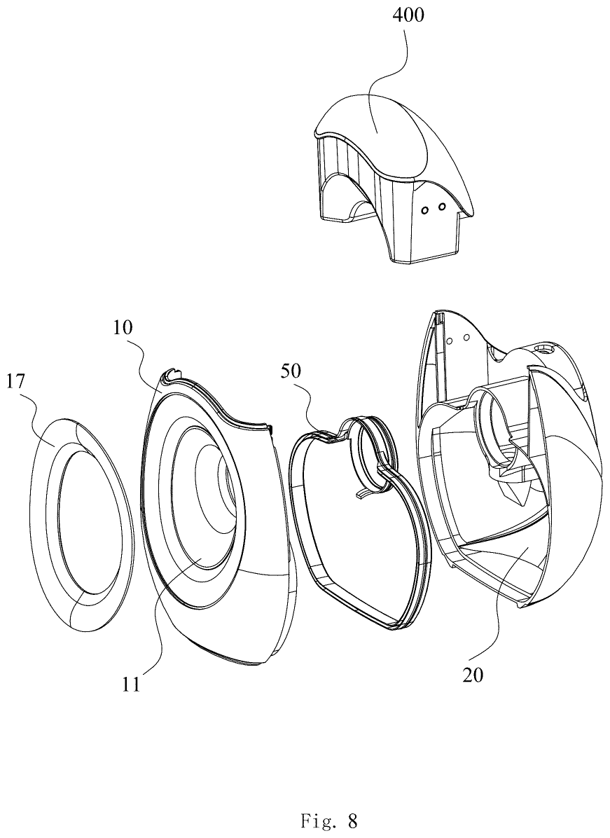

FIG. 9 is a schematic view of a combination of a front shell and a negative pressure chamber of the breast pump according to another embodiment in the present disclosure.

FIG. 10 is an exploded structural diagram of FIG. 9.

FIG. 11 is a schematic structural view of the rear shell of the breast pump according to another embodiment in the present disclosure.

FIG. 12 is a disassembled structure diagram of the breast pump according to an embodiment in the present disclosure.

FIG. 13 is a schematic exploded view of a host module of the breast pump according to an embodiment in the present disclosure.

### EMBODIMENTS

Embodiments of the present disclosure are described in detail below, and examples of the embodiments are shown in the attached drawings, in which the same or similar labels represent the same or similar elements or elements with the same or similar function. The embodiments described below by reference to the attached drawings are exemplary and are intended to explain embodiments of the present disclosure and cannot be understood as limits in the present disclosure.

In the description of the embodiments of the present disclosure, it should be understood that if the embodiments involve directional indications, such as the “upper”, “lower”, “left”, “right”, “front”, “rear”, “internal” and “external”, etc. indicating orientation or location relationship, is based on the orientation or position relationship shown in the figures, it is only for describing the embodiments in the present disclosure and simplifying the description, rather than indicating or implying the device or the element that must have a specified azimuth, constructed and operated, it cannot be understood as a limitation on the present disclosure.

In the description of the present disclosure, several meanings are one or more, multiple meanings are two or more, greater than, less than, more than, etc. are understood as not including the original number, and above, below, within, etc. are understood as including the original number. If there is a description that the first and second are only to distinguish technical features, and cannot be understood as indicating or implying relative importance or implying the quantity or sequence of the indicated technical features.

In the description of the present disclosure, unless otherwise specified, the terms “setting”, “installation”, “connection”, etc. should be understood in a broad sense. Those skilled in the art can reasonably determine the specific meanings of the above words in the present disclosure based on the specific content of the technical solution.

Hereinafter, a breast pump according to embodiments in the present disclosure will be described with reference to the accompanying drawings. For ease of understanding and description, a side of the breast pump that is close to a breast is defined as the front side of the breast pump, and a side opposite to the front side of the breast pump is defined as the rear side of the breast pump.

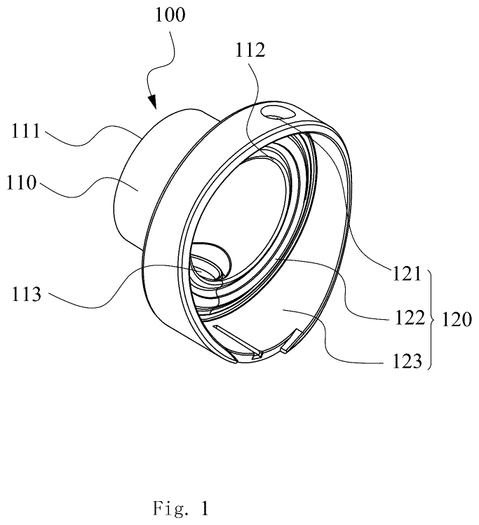

Referring to FIG. 1, the present disclosure discloses a connector 100 of a breast pump, which has a connecting barrel 110 with a front opening 111 and a rear opening 112 opposite to each other. The front opening 111 faces and connects the below trumpet-shaped cavity 11. The periphery of the rear opening 112 extends laterally to form a rear chamber 120, used for accommodating the below negative pressure chamber 200. The connecting barrel 110 is in communication with the rear chamber 120. The bottom surface of the connecting barrel 110 is provided with a first through-hole 113, used for being in communication with the below storage chamber 40. The rear chamber 120 is provided with a second through-hole 121, used for connecting the below host module 400. With the connector 100, the trumpet-shaped cavity 11, the negative pressure chamber 200, the host module 400, and the storage chamber 40 are respectively integrated in the front, rear, upper, and lower direction of the breast pump, thereby facilitating the connection between various components, reducing an additional connection structure, enabling the following breast pump to be light and precise in structure, and being convenient to use.

In addition, the host module 400 and the storage chamber 40 are respectively arranged in the upper direction and the lower direction of the connector, so that the thickness of the breast pump in the front-rear directions is reduced, the structure thereof is compact, the parts are evenly distributed, the layout is reasonable, and the shape is small and beautiful. The reduction of the thickness of the breast pump makes the chest shape of the occupational mother not much the same when she wears the breast pump or not, the breast pump is aesthetic and does not leak the privacy of the occupational mother, and is very practical.

In addition, the rear chamber 120 includes a corrugated plate 122 connecting the rear opening 112, a side wall 123 perpendicularly extends from the periphery of corrugated plate 122 such that the rear chamber 120 is in a groove shape; the second through-hole 121 is provided at the side wall 123, preferably in the top portion of the side wall 123.

The present disclosure also provided the breast pump having the above connector 100. The breast pump includes a front shell 10 and a rear shell 20 which are connected in a matching manner and enclose an inner cavity of the breast pump. The front surface of the front shell 10 is provided with a trumpet-shaped cavity 11, and the front opening 111 faces the trumpet-shaped cavity 11.

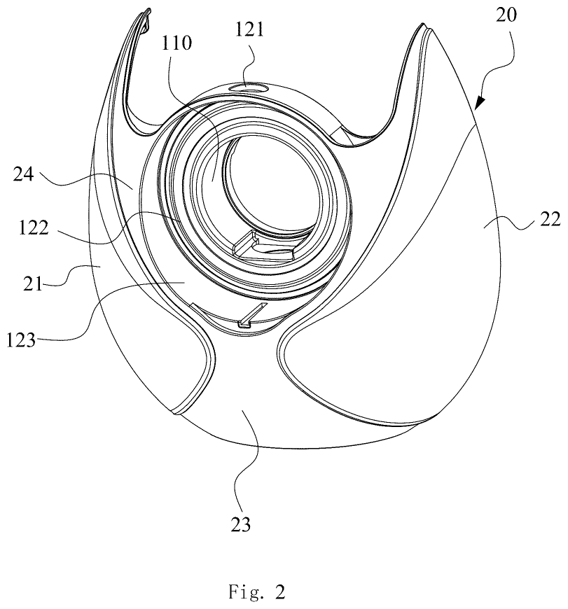

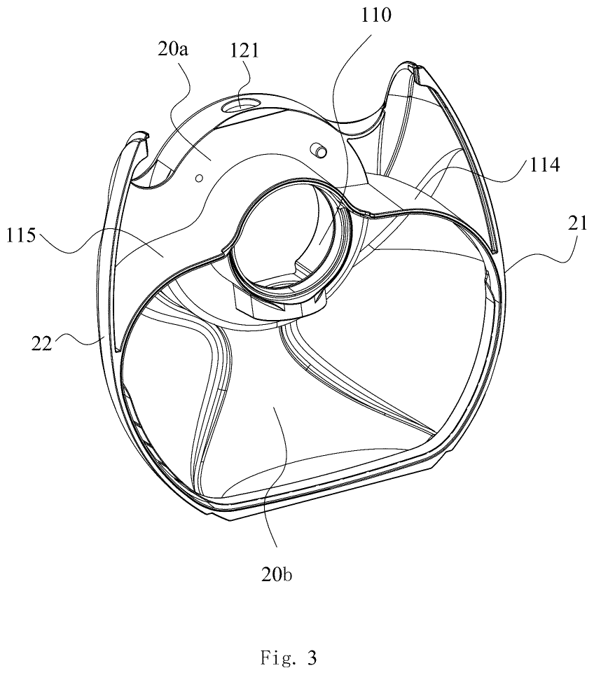

In one embodiment in the present disclosure, referring to FIGS. 2-3, the connector 100 is provided in the middle of the rear shell 20 and integrally formed with the rear shell 20. Specifically, the rear shell 20 has a rear-shell left portion 21, a rear-shell right portion 22, and a rear-shell middle portion 23 connecting the rear-shell left portion 21 and the rear-shell middle portion 22. The side wall 123 of the rear chamber 120 connects the rear-shell middle portion 123. The rear-shell left portion 21 and the rear-shell right portion 22 are located at two sides of the connecting barrel 110.

Preferably, the rear-shell middle portion 23 is provided with an empty place 24 at a position directly facing the rear chamber 120 so that the negative pressure chamber 200 contained in the rear chamber 120 is exposed out of the rear shell 20, thereby fascinating to mount and detach the negative pressure chamber 200.

Referring to FIG. 3, a first partition 114 and a second partition 115 extend symmetrically from the left and right side walls of the connecting barrel 110, the first partition 114 is connected to the rear-shell left portion 21, and the second partition 115 is connected to the rear-shell right portion 22 so that the rear shell 20 is divided into an upper part 20a of the rear shell and a lower part 20b of the rear shell.

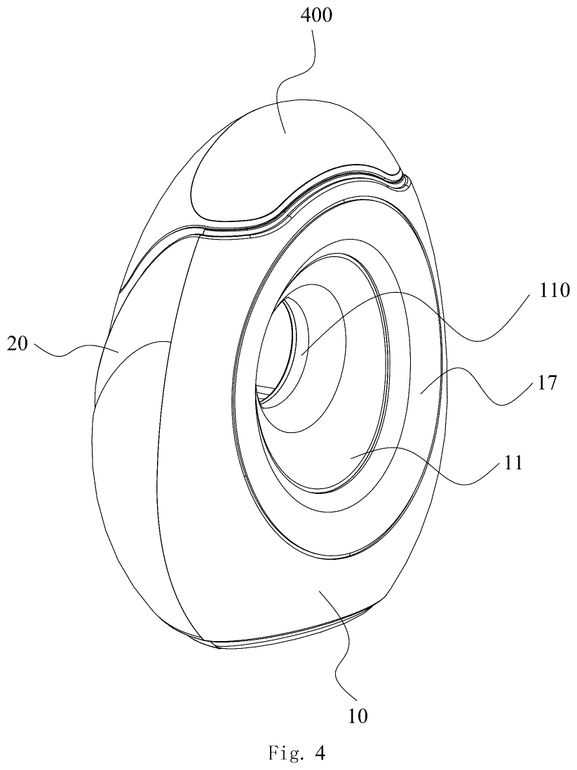

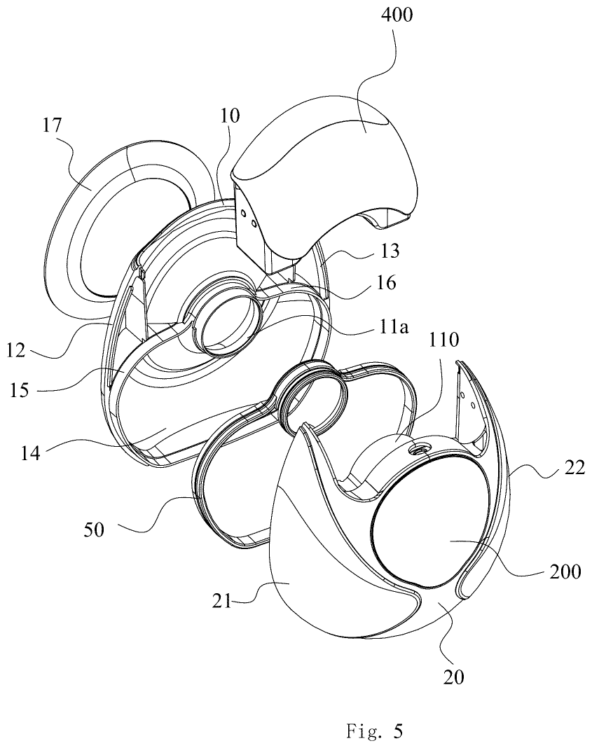

Referring to FIGS. 4-5, the rear chamber 120 accommodates the negative pressure chamber 200, the negative pressure chamber 200 includes a silica gel deformation part 210, a rear cover in a groove-shaped rear cover 220. The silica gel deformation part connects the corrugated plate 122 of the rear chamber 120, and the groove-shaped rear cover 220 attaches to the side wall 123 of the rear chamber 120, thereby mounting the negative pressure chamber 200 in the rear chamber 120. The groove-shaped rear cover 220 is provided with a third through-hole 221 at a position directly opposite to the second through-hole 121. The host module 400 is disposed at the upper part 20a of the rear shell 20. The host module 400 alternately inflates or deflates the negative pressure chamber 200 through one gas pipe 401 passing through the second through-hole 121 and the third through-hole 221. The gas pipe 401 is connected to the third through-hole 221 in a sealing manner. The host module 400, the gas pipe 401, and the negative pressure chamber 200 form a gas pathway.

The central portion 211 of the silica gel deformation 210 protrudes into the rear opening 112 of the connecting barrel 110. When the host module 400 inflates to the negative pressure chamber 200, the gas amount in the negative pressure chamber 200 increases, and a positive pressure is generated, then the central portion 211 of the silica gel deformation 210 expands towards the front opening 111 of the connecting barrel 110 to reduce the space in the trumpet-shaped cavity 11 and the connecting barrel 110. At this time, if the front shell 10 hermetically attaches to the mother's breast, the gas in the space between the trumpet-shaped cavity 11 and the connecting barrel 110 is compressed, and the pressure therein is increased. Then the host module 400 stops inflating to the negative pressure chamber 200 but deflates from the negative pressure chamber 200 so that the gas amount in the negative pressure chamber 200 is reduced, and a negative pressure is generated. The silica gel deformation 210 resets under the action of its elastic force, so that the space in the trumpet-shaped cavity 11 and the connecting barrel 110 is increased, and the pressure thereof is reduced. By reciprocating in this way, the effect of simulating baby sucking is generated, and the breast is driven to secrete the milk.

Referring to FIGS. 4-7, the front shell 10 is provided with the trumpet-shaped cavity 11 passing through the front shell 10, and the small opening end 11a of the trumpet-shaped cavity 11 connects the front opening 111 of the connecting barrel 110.

The front shell 10 has a front-shell left portion 12, the front-shell right portion 13, and a front-shell middle portion 14 connecting the front-shell left portion 12 and the front-shell right portion 13. The trumpet-shaped cavity 11 is provided in the front-shell middle portion 14. The front shell 10 extends from the left and right sides of the small opening end 11a of the trumpet-shaped cavity 11 to form a front-left edge 15 connecting the front-shell left portion 12 and a front-right edge 16 connecting the front-shell right portion 13 respectively. The rear-shell left portion 15 is connected to the first partition 114, and the rear-shell right portion 16 is connected to the second partition 115 so that the inner cavity of the breast pump is divided into the upper mounting chamber 30 and the lower storage chamber 40. The upper mounting chamber 30 is used for accommodating the host module 400, and the storage chamber 40 is used for collecting and storing the milk.

Preferably, the sealing ring 50 is provided at the joint of the front shell 10 and the rear shell 20, connecting the front-left edge 15 and the first partition 114, the front-right edge 16 and the second partition 115, and the small opening end 11a of the trumpet-shaped cavity 11 and the front opening 111 of the connecting barrel 110, and another joint of the edges of the front shell 10 and the rear shell 20 in a sealed manner.

Specifically, the sealing ring 50 has a ring 51 and two strips 52 arranged at two sides of the ring 51, the ring 51 is hermetically connected to the small opening end 11a of the trumpet-shaped cavity 11 and the front opening 111 of the connecting barrel 110. The two strips 52 are respectively connected to the front-left edge 15 and the first partition 114, and the front-right edge 16 and the second partition 115.

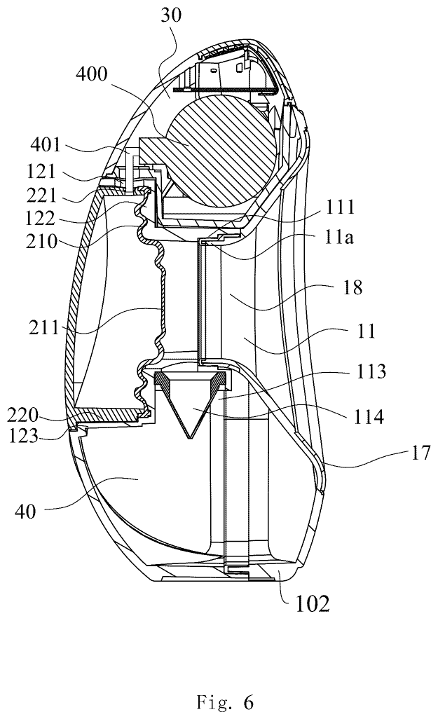

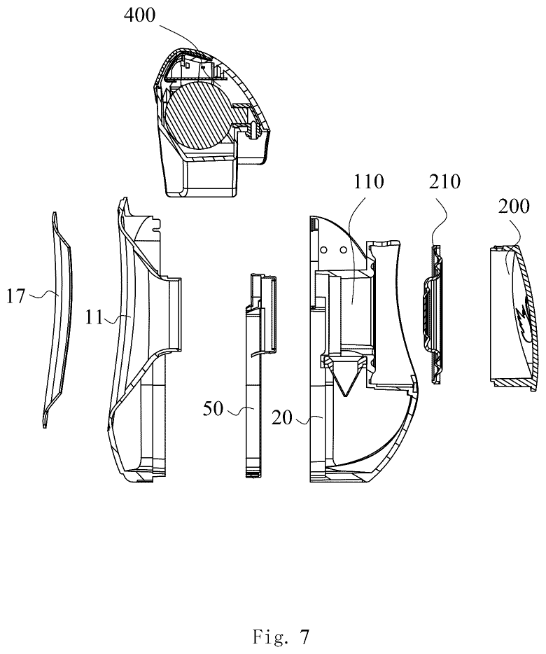

Referring to FIGS. 6-7, the connecting barrel 110 is in communication with the storage chamber 40 via the first through-hole 113, a one-way valve 114 is provided at the first through-hole 113, the milk flows from the trumpet-shaped cavity 11 into the connecting barrel 110, and then passes through the one-way valve 114 and enters the storage chamber 40. The one-way valve 114 controls the milk to only flow into the storage chamber 40 and not flow out thereof. All of the trumpet-shaped cavity 11, the connecting barrel 110, the first through-hole 113, and the storage chamber 40 form a milk pathway. In the present disclosure, the one-way valve 114 is provided at the first through-hole 113 and is located at the top of the storage chamber 40, and the milk passes through the one-way valve and enters the storage chamber 40 depending on the gravity thereof, the flow of the milk is smooth and does not remain, which helps to secrete the milk, and solves the pressure caused by the fact that the milk enters from the bottom of the storage chamber 40 and pushes the milk in the storage chamber 40 upwards in the prior art.

The sealing ring 50, the front-left edge 15, the first partition 114, the front-right edge 16, and the second partition 115 isolate the milk pathway and the gas pathway of the breast pump, which do not affect each other, and the milk flows smoothly without remaining.

Referring to FIG. 5, a pad 17 is attached to the trumpet-shaped cavity 11 so that the trumpet-shaped cavity is soft and comfortable when being attached to the mother's breast and has good sealing performance.

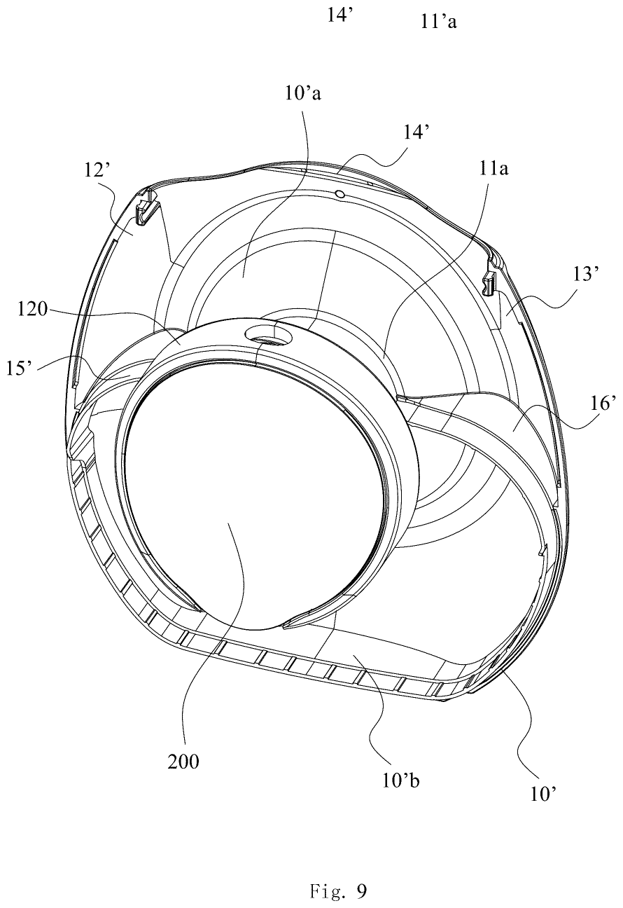

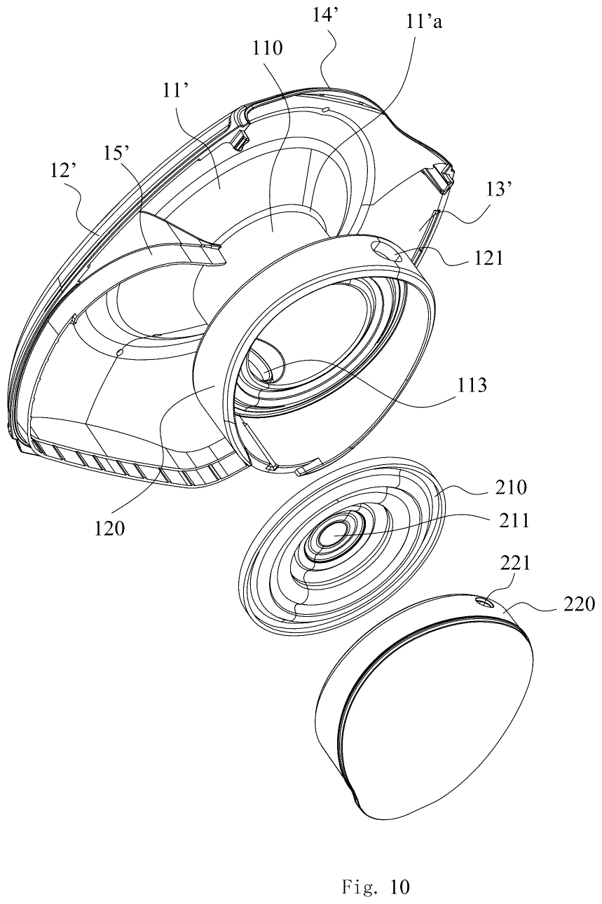

Referring to FIGS. 9 and 10, which show another embodiment of the breast pump in the disclosure, the connector 100 is disposed in the middle of the front shell 10′. Specifically, the front shell 10′ has a front-shell left portion 12′, the front-shell right portion 13′, and a front-shell middle portion 14′ connecting the front-shell left portion 12′ and the front-shell right portion 13′. The front-shell middle portion 14′ is provided with the trumpet-shaped cavity 11, and the small opening end 11a of the trumpet-shaped cavity 11 and the front opening 111 of the connecting barrel 110 is integrally formed.

The front shell 110 extends from the left and right sides of the small opening end 11a of the trumpet-shaped cavity 11 to form a front-left edge 15′ connecting the front-shell left portion 12′ and a front-right edge 16′ connecting the front-shell right portion 13′ respectively, thereby dividing the front shell into a front shell upper portion 10a and a front shell lower portion 10b.

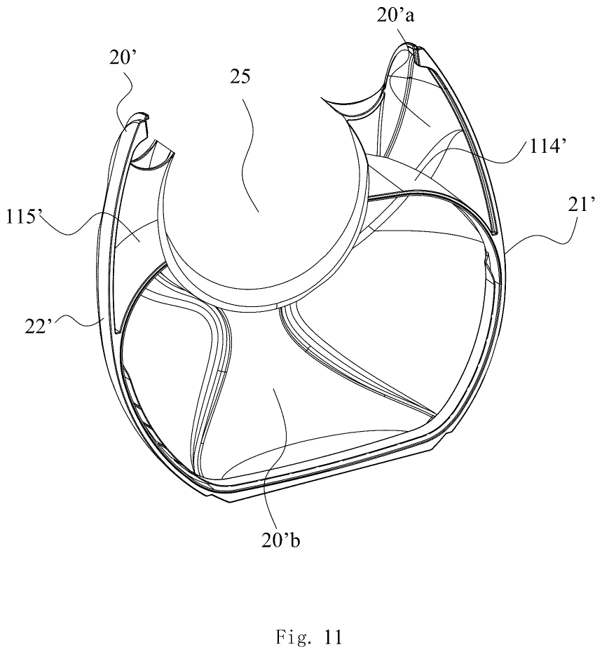

Referring to FIG. 11, the breast pump further includes the rear shell 20′ matching the front shell 10′, the rear shell 20′ has a rear-shell left portion 21′, a rear-shell right portion 22′, and a rear-shell middle portion 23′ connecting the rear-shell left portion 21′ and the rear-shell middle portion 22′. The rear shell 20′ is provided with a fitting groove 25 at a position directly facing the rear chamber 120, and the rear chamber 120 is partially accommodated in the fitting groove 25. The first partition 114′ and the second partition 115′ extend symmetrically from the left and right side walls of the fitting groove 25. The first partition 114′ is connected to the rear-shell left portion 21′, and the second partition 115′ is connected to the rear-shell right portion 22′, so that the rear shell 20′ is divided into an upper part 20′a of the rear shell and a lower part 20′b. The front-left edge 15′ connects the first partition 114′ and the front-right edge 16′ connects the second partition 115′ such that the inner cavity of the breast pump is divided into the upper mounting chamber 30 and the lower storage chamber 40.

In another embodiment in the present disclosure, referring to FIGS. 3-5 and 12, the breast pump includes the shell 1 composed of the front shell 10 and the rear shell 20, and the host module 400. The host module 400 is detachable with the shell 1. The electrical elements are arranged in the host module 400, and when the breast pump is cleaned, the electric elements are separated from the shell, making cleaning safer. The shell 1 is provided with the storage chamber 40, the negative pressure chamber 200, and the trumpet-shaped cavity 11 therein, and the trumpet-shaped cavity 11 and the storage chamber 40 are in a unidirectional communication to prevent the milk in the storage chamber 40 from flowing backward. The negative pressure chamber 200 is located at one side of the trumpet-shaped cavity 11. When the breast pump is in use, the trumpet-shaped cavity 11 is hermetically attached to a mother's breast, and the negative pressure chamber 200 is inflated or deflated alternatively to enable the trumpet-shaped cavity 11 in a positive pressure state or a negative pressure state, thereby simulating an infant sucking action, so as to suck mother's milk.

The pressure in the negative pressure chamber 200 is adjusted by the host module 400. The host module 400 comprises a driving assembly 410 and a power supply assembly 420. The driving assembly 410 is electrically connected to the power supply assembly 420. The suction port of the driving assembly 410 is in communication with the negative pressure chamber 200. The driving assembly 410 adopts an air pump structure to realize an inflating and deflating function.

In the host module 400, the driving assembly 410 is arranged in the middle of the host module 400, and the power supply assembly 420 is arranged in a symmetrical layout, that is the power supply assembly 420 is divided into two parts and symmetrically arranged at two sides of the driving component 410. Since the driving assembly 410 and the power supply assembly 420 occupy most of the weight of the host module 400, the driving assembly 410 and the power supply assembly 420 are reasonably distributed, so that the weight distribution of the host module 400 is more uniform. When mom wears the breast pump, the weight of the host module 400 is evenly distributed above the breast, thus avoiding the situation that the breast pump slips or the breast pump pulls the breast due to uneven weight distribution thereof, and protecting the breast.

The storage chamber 40 is provided with a milk channel 41, extending to the top of the shell 1. When the host module 400 is mounted on the shell 1, the bottom of the host module 400 blocks the port of the milk channel. When the driving assembly 410 deflates from the negative pressure chamber 200, the volume of the negative pressure chamber 200 decreases, the volume of the space between the trumpet-shaped cavity 11 and the negative pressure chamber 200 increases, and a negative pressure is formed in the trumpet-shaped cavity 11. At this time, the milk is sucked out. When the driving assembly 410 inflates the negative pressure chamber 200, the volume of the negative pressure chamber 200 increases, the volume of the trumpet-shaped cavity 11 decreases, and a positive pressure is formed in the trumpet-shaped cavity 11. The milk flows into the storage chamber 40. And the milk channel is in communication with the outside so that the internal pressure of the storage chamber 40 is consistent with the external atmospheric pressure.

Specifically, after the main module 400 and the shell 1 are assembled, a gap is reserved between the port of the milk channel 41 and the bottom of the main module 400. When the breast pump is in a storage state or in a use state, the host module 400 can block the opening of the milk channel 41, so as to prevent external contaminants from entering the storage chamber 40 from the port of the milk channel 41, and can also keep the milk channel 41 in communication with the outside, thereby ensuring that the internal pressure and the external pressure of the milk chamber 40 are equal, and enabling the milk to flow into the storage chamber 40 successfully. If the milk channel 41 is in a closed state, as the amount of the milk in the storage chamber 40 increases, the pressure in the storage chamber 40 increases, the pressure difference between the trumpet-shaped cavity 11 and the storage chamber 40 decreases, and the milk is unlikely to flow into the storage chamber 40, thereby affecting the working efficiency of the breast pump.

The breast pump in the present disclosure optimizes the structure of the milk channel 41 of the storage chamber 40 so that the breast pump can ensure the hygiene of the storage chamber 40 in any state, thereby reducing pollution. The port position of the milk channel 41 is optimized, and the length of the milk channel 41 is shortened, so that the milk channel 41 does not occupy the lateral space of the breast pump, thereby being beneficial to reducing the thickness of the breast pump, and being more comfortable to wear. The driving assembly 410 and the power supply assembly 420 are symmetrically arranged in the host module 400, so that the weight distribution of the host module 400 is more uniform, the breast pump is more stably worn and won't pull the breast, thereby protecting the breast.

In some embodiments of the present disclosure, as shown in FIGS. 3 and 6, the front shell 10 includes the connecting barrel 110, a trumpet-shaped cavity 11, and a pad 17, the connecting barrel 110 is provided at one side of the negative pressure chamber 200, the small opening end of the trumpet-shaped cavity 11 is in communication with the connecting barrel 110, and the surface of the trumpet-shaped cavity 11 is provided with the pad 17.

Specifically, the connecting barrel 110, the trumpet-shaped cavity 11, and a pad 17 are sequentially arranged on the front shell 10, the connecting barrel 110 is located at the small opening end of the trumpet-shaped cavity 11, and the pad 17 is made of a silicone material and is attached to the surface of the trumpet-shaped cavity 11. When the trumpet-shaped cavity 11 is attached to the breast, the pad 17 is attached to the breast, which can make the sealing better, thereby avoiding the effect of air leakage on the breast absorption effect. The pad 17 made of a silicone material can also reduce the sensitivity of the breast and is more comfortable to wear.

According to some embodiments in the disclosure, referring to FIG. 6, the contour of the trumpet-shaped cavity 11 is provided in a breast-like manner, the pad 17 is provided at the opening of the trumpet-shaped cavity 11, and the pad 17 is used for attaching to the breast.

Specifically, the contour of the trumpet-shaped cavity 11 and the contour of the breast match each other, and when the breast pump is attached to the breast, the pressing feeling of the trumpet-shaped cavity 11 on the breast can be reduced. Compared with the conventional shape of the existing breast pump, the trumpet-shaped cavity 11 of the present embodiment reduces the compression on the breast using bionic shaping, can reduce the depth of the trumpet-shaped cavity 11, and reduces the thickness of the breast pump. The bionic shape can also improve the fitting area of the trumpet-shaped cavity 11 and the breast, and has better fitting sealing performance. In addition, the weight of the breast pump can be distributed on the surface of the breast, thereby preventing the breast from being deformed due to concentrated stress.

In some embodiments of the present disclosure, as shown in FIG. 6, a silica gel deformation portion 210 is disposed between the connecting barrel 110 and the negative pressure chamber 200, and a surface contour of the attaching portion of the trumpet-shaped cavity 11 with the silica gel deformation portion 210 and a surface contour of the silica gel deformation portion 210 match each other.

Specifically, the cross-section of the silica gel deformation part 210 is in a folded edge shape so that it is easily deformed and the deformation volume is relatively large, which increases the volume change of the space among the negative pressure chamber 200, the connecting barrel 110 and the trumpet-shaped cavity 11, in other words, increasing the pressure change in the space, thereby improving the milk sucking efficiency. The silica gel deformation part 210 is made of a silica gel material and has a better elastic deformation effect. In order to make the attachment and sealing between the trumpet-shaped cavity 11 and the silica gel deformation part 210 better, the surface contour where the trumpet-shaped cavity 11 attaches to the silica gel deformation part 210 matches the surface contour of the silica gel deformation part 210, thereby increasing the contact area, and having a better sealing performance. The working principle that the silica gel deformation part 210 is deformed by the pressure change of the negative pressure chamber 200 is well known to those skilled in the art, and is not described in detail in this embodiment.

In some embodiments of the present disclosure, as shown in FIG. 3, a one-way valve 114 is provided at the bottom of the connecting barrel 110, and the connecting barrel 110 is in unidirectional communication with the storage chamber 40.

Specifically, one-way valve 114 is made of silicone material and is in a closed state under normal conditions. When the volume of the negative pressure chamber 200 increases, the volume of the space among the negative pressure chamber 200, the connecting barrel 110, and the trumpet-shaped cavity 11 decreases and the pressure therein increases, pushing the one-way valve 114 to open. Then, under the action of gravity, the breast milk enters the storage chamber 40 from the one-way valve 114. When the volume of the negative pressure chamber 200 is reduced, the volume of the space among the negative pressure chamber 200, the connecting barrel 110, and the trumpet-shaped cavity 11 increases and the pressure therein reduces, when the pressure in the storage chamber 40 is greater than that in the space, the one-way valve 114 is restored and closed. The above process is repeated in the breast pump, allowing the breast milk to be stored in the storage chamber 40 after each suction and effectively preventing the back-flow of breast milk in the storage chamber 40.

In some embodiments of the present disclosure, as shown in FIGS. 4-6, the shell 1 comprises the front shell 10 and the rear shell 20, the trumpet-shaped cavity 11 is located at the front shell 10, the connecting barrel 110 is located at the rear shell 20, and the storage chamber 40 is composed of a combination of the front shell 10 and the rear shell 20.

Specifically, the shell 1 of the present disclosure is composed of the front shell 10 and a rear shell 20, which are engaged through a buckle structure and the storage chamber 40 is located at the lower portion of shell 1. The shell 1 facilitates disassembly and cleaning of the interior of the storage chamber 40, which reduces cleaning difficulty and makes it easier to clean hygiene blind spots.

In some embodiments of the present disclosure, as shown in FIGS. 5-6, a scaling ring 50 is provided at the joints between the front shell 10 and the rear shell 20. The scaling ring 50 seals the connection gap between the trumpet-shaped cavity 11 and the connecting barrel 110, as well as the connection gap between the storage chamber 40.

Specifically, due to the storage chamber 40 is formed by the connection of the front shell 10 and the rear shell 20, there exists a connecting gap between the front shell 10 and the rear shell 20. And the sealing performance is unstable. Therefore, a sealing ring 50 is used to fill the connection gaps and improve the sealing performance of the storage chamber 40 to prevent liquid leakage. The connection gap between the trumpet-shaped cavity 11 and the connecting barrel 110 also can lead to a decrease in the working efficiency of the breast pump because of a decrease in sealing performance. The sealing ring 50 can simultaneously fill the connection gap between the trumpet-shaped cavity 11 and the connecting barrel 110, further improving the sealing performance of the breast pump.

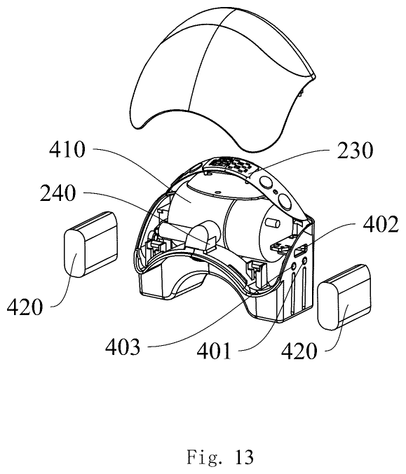

In some embodiments of the present disclosure, as shown in FIGS. 6 and 13, the host module 400 is provided with a negative pressure hole 401, and the suction port of the driving assembly 410 is connected to the negative pressure hole 401 through a silicone tube. When the host module 400 is connected to the shell 1, the negative pressure hole 401 is tightly connected to the second through-hole 121 of the negative pressure chamber 200.

Specifically, the air hole of the negative pressure chamber 200 adopts a sinking hole structure, while the negative pressure hole 401 adopts a convex hole structure. When the negative pressure chamber 200 is connected to the negative pressure hole 401, the negative pressure hole 401 is closely connected to the air hole, allowing the driving assembly 410 to change the internal pressure of the negative pressure chamber 200 and achieve the simulated sucking action of the breast pump. The periphery of the negative pressure hole 401 is equipped with a silicone sealing ring, which can improve the sealing performance between negative pressure hole 401 and the negative pressure chamber 200.

Specifically, the air hole of the negative pressure chamber 200 adopts a sinking hole structure, while the negative pressure hole 401 adopts a convex hole structure. When the negative pressure chamber 200 is connected to the negative pressure hole 401, the negative pressure hole 401 is closely connected to the air hole, allowing the driving assembly 410 to change the internal pressure of the negative pressure chamber 200 and achieve the simulated sucking action of the breast pump. The periphery of the negative pressure hole 401 is equipped with a silicone sealing ring, which can improve the sealing performance between negative pressure hole 401 and the negative pressure chamber 200.

Specifically, the host module 400 also includes a charging interface 402, which is set on the other side of the suction port of the driving assembly 410, so that the silicone tube and the charging interface 402 are located on both sides of the driving assembly 410, balancing the weight distribution of the host module 400.

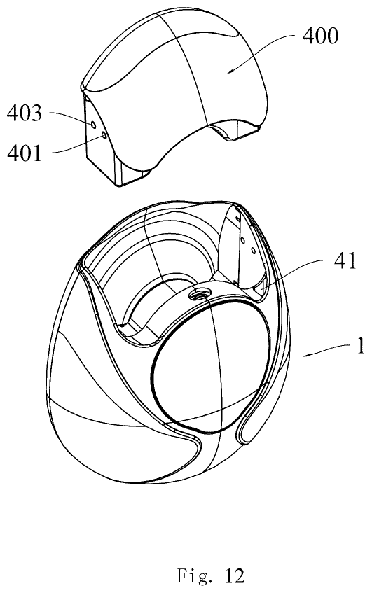

In some embodiments of the present disclosure, as shown in FIG. 12, the host module 400 and the shell 1 are engaged through an elastic structure. Specifically, a pin 403 is provided at the host module 400 and adopted to connect the host module 400 and the shell 1, the connection is detachable. When the host module 400 is connected to the shell 1 in place, the pin 403 matches with the inner wall positioning groove of the shell 1, and the pin 403 pops out into the positioning groove and collides to produce a sound, providing positive feedback to the user for connection.

It should be understood that the elastic structure used for connecting the host module 400 and the shell 1 is not the only implementation method. In other embodiments, the magnetic suction structure can also be used, which can achieve the effect of automatic guidance connection. The present disclosure does not elaborate on the connecting structure between the host module 400 and the shell 1. It should be understood that, without departing from the basic concept of the present disclosure, the flexible transformation of the connecting structure between the host module 400 and the shell 1 should be considered within the protection scope limited by the claim.

In the description of this specification, the reference terms “one embodiment”, “some embodiments”, “illustrative embodiments”, “examples”, “specific examples”, or “some examples” refer to specific features, structures, materials, or features described in conjunction with the embodiments or examples included in at least one embodiment or example of the present disclosure. In this specification, the illustrative expressions of the above terms may not necessarily refer to the same embodiments or examples. Moreover, the specific features, structures, materials, or features described can be combined appropriately any one or more embodiments or examples.

Although embodiments of the present disclosure have been shown and described, those skilled in the art may understand that various changes, modifications, substitutions, and variations can be made to these embodiments without departing from the principles and objectives of the present disclosure, and the scope of the present disclosure is limited by the claims and their equivalents.

1. A connector, comprising a connecting barrel with a front opening and a rear opening opposite to each other, a periphery of said rear opening extends outward to form a rear chamber, said connecting barrel is in communication with said rear chamber;
a bottom surface of said connecting barrel is provided with a first through-hole, and said rear chamber is provided with a second through-hole.

2. The connector according to claim 1, wherein said rear chamber comprises a corrugated plate connecting said rear opening, a side wall perpendicularly extends from a periphery of said corrugated plate such that said rear chamber is in a groove shape; and
said second through-hole is formed in a top portion of said side wall.

3. A breast pump, comprising:
said connector according to claim 2,
a front shell and a rear shell connected in a matching manner,
a front surface of said front shell is provided with a trumpet-shaped cavity, and said front opening faces said trumpet-shaped cavity.

4. The breast pump according to claim 3, wherein said rear shell has a rear-shell left portion, a rear-shell right portion, and a rear-shell middle portion connecting said rear-shell left portion and said rear-shell middle portion; said connector is integrally formed with said rear shell and provided in said rear-shell middle portion;
said side wall of said rear chamber connects said rear-shell middle portion; and
said rear-shell left portion and said rear-shell right portion are located at two sides of said connecting barrel.

5. The breast pump according to claim 4, wherein said rear-shell middle portion is provided with an empty place at a position directly facing said rear chamber.

6. The breast pump according to claim 4, wherein a first partition and a second partition extend symmetrically from left and right side walls of said connecting barrel, said first partition is connected to said rear-shell left portion, and said second partition is connected to said rear-shell right portion so that said rear shell is divided into an upper part of said rear shell and a lower part of said rear shell.

7. The breast pump according to claim 6, wherein said trumpet-shaped cavity passes through said front shell with a small opening end thereof connecting said front opening of said connecting barrel;
said front shell has a front-shell left portion, a front-shell right portion, and a front-shell middle portion;
said trumpet-shaped cavity is located in said front-shell middle portion;
said front shell extends from left and right sides of said small opening end of said trumpet-shaped cavity to from a front-left edge connecting said front-shell left portion and a front-right edge connecting said front-shell right portion respectively; and
said front-left edge connects said first partition and said front-right edge connects said second partition such that an inner cavity of said breast pump is divided into an upper mounting chamber and a lower storage chamber.

8. The breast pump according to claim 7, wherein said sealing ring is provided at a joint of said front shell and said rear shell, connecting said front-left edge and said first partition, said front-right edge and said second partition, and said small opening end and said connecting barrel in a sealed manner.

9. The breast pump according to claim 3, wherein said front shell has a front-shell left portion, a front-shell right portion, and a front-shell middle portion connecting said front-shell left portion and said front-shell right portion;
said trumpet-shaped cavity is provided in said front-shell middle portion, and said small opening end of said trumpet-shaped cavity and said front opening of said connecting barrel is integrally formed.

10. The breast pump according to claim 9, wherein further comprises a rear shell matching said front shell, said rear shell is provided with a fitting groove at a position directly facing said rear chamber, and said rear chamber is partially accommodated in said fitting groove.

11. The breast pump according to claim 9, wherein said front shell extends from said left and right sides of said small opening end of said trumpet-shaped cavity to form a front-left edge connecting said front-shell left portion and a front-right edge connecting said front-shell right portion respectively;
a first partition and a second partition extend symmetrically from left and right side walls of said fitting groove, said first partition is connected to said rear-shell left portion, and said second partition is connected to said rear-shell right portion, said front-left edge connects said first partition, said front-right edge connects said second partition so that an inner cavity of said breast pump is divided into an upper mounting chamber and a lower storage chamber.

12. The breast pump according to claim 3, further comprising a host module, detachably connected to said front shell and said rear shell, said host module comprises a central drive assembly and a symmetrical power supply assembly, said symmetrical power supply assembly is electrically connected to said drive assembly, and a suction port of said drive assembly is in communication with a negative pressure chamber arranged in said rear chamber.

13. The breast pump according to claim 3, wherein a top part of said front shell or said rear shell is provided with a milk channel, and an host module is arranged on said front shell and said rear shell; when said host module, said front shell, and said rear shell are assembled, a bottom portion of said host module shields a port of said milk channel.
